(19) United States
(12) Patent Application Publication
(10) Pub. No.: US 2011/0111986 A1
(43) Pub. Date: May 12, 2011

(54) NOVEL SACCHARIDE PRIMER AND USE THEREOF

(75) Inventors: Toshinori Sato, Kanagawa (JP); Hideki Nakajima, Tokyo (JP)

(73) Assignee: KEIO UNIVERSITY, Minato-ku, Tokyo (JP)

(21) Appl. No.: 12/808,811

(22) PCT Filed: Dec. 18, 2008
(86) PCT No.: PCT/JP2008/073079

§ 371 Date: Nov. 4, 2010

(30) Foreign Application Priority Data
Dec. 18, 2007 (JP) 2007-325804

(51) Int. Cl. C40B 40/12; C07H 15/10; C12P 19/44; C07H 1/00; C40B 50/14; C07K 1/107; C07K 2/00

(52) U.S. Cl. 506/19; 536/4.1; 435/74; 536/18.5; 506/30; 530/322

(57) ABSTRACT

The present invention can provide a novel saccharide primer, which makes it possible to efficiently synthesize a sugar chain, and a method for using the primer. According to the present invention, the saccharide primer of the present invention is a saccharide primer having an alkenyl chain that has one or more double bonds in a position other than the alkyl chain terminal, and represented by the following general formula (I):

Lac-O-Ck:m(n) General formula (I)

In the general formula (I), Ck represents an alkenyl chain, m represents a total number of double bonds, and n represents a position of the double bond in the alkenyl chain. Here, k is preferably an integer in the range of 8 to 14, and particularly 12 or 13. In addition, when m is 1, n is an integer satisfying 1≦n≦k−2; and when m satisfies 2≦m≦k−1, n is a plurality of integers satisfying 1≦n≦k−1.

An example of the primer is the following general formula (II):

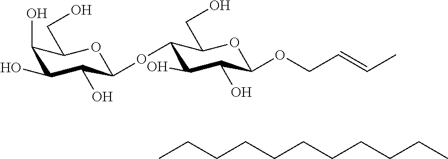

A sugar chain synthesized by using the saccharide primer of the present invention can be used for producing a sugar chain array, for synthesizing a glycopeptide, and the like.

### TECHNICAL FIELD

The present invention relates to a novel saccharide primer and a method for using the primer.

### BACKGROUND ART

Sugar chains play an important role as signals or marker molecules in ontogenesis, cell differentiation, cell proliferation, apoptosis, histogenesis, immunity, blood group antigens, receptors for toxins or viruses, canceration, diseases, expression of cellular functions, cell-to-cell interactions and the like. Therefore, the demand for sugar chains is increasing in the field of biotechnology and in the development of pharmaceuticals. In particular, sugar chains are expected to be applied to pharmaceuticals, diagnostic drugs, glycochips, and the like.

Recently, a saccharide primer capable of producing a sugar chain by using a cell function has been developed (Japanese Unexamined Patent Application Publication No. 2000-247992 and Japanese Unexamined Patent Application Publication No. 2005-117918). In general, saccharide primers have a structure of sugar-alkyl, sugar-amide-alkyl or sugar-amino acid-alkyl. However, saccharide primers having a modified alkyl group have been developed, and for example, the following saccharide primers are described: a saccharide primer in which an azide group as a functional group is introduced into a free terminal (Japanese Unexamined Patent Application Publication No. 2000-247992), a saccharide primer in which a polymerizable vinyl group is introduced into a free terminal (Hatanaka, K., Kobayashi, M., and Kasuya, M. C., Seisan Kenkyu 56, 234-238 (2004)), and a saccharide primer having an azide group in an alkyl group (Kasuya, M. C. Z., Kobatashi, M., Watanabe, Y., Sato, T., and Hatanaka, K., Chemistry & Biodiversity 2, 1063-1078 (2005)).

However, these saccharide primers have a long alkyl chain between a sugar chain and a functional group, and therefore, show high hydrophobicity. Consequently, sugar chains synthesized by using these saccharide primers bind to other substances nonspecifically, and it is difficult to use such sugar chains for the purpose of experiments, and the like.

### DISCLOSURE OF INVENTION

### Problems to be Solved by the Invention

Therefore, there has been a need to develop a saccharide primer which makes it possible to efficiently synthesize sugar chains in cells and to easily use the synthesized sugar chains for various purposes.

An object of the present invention is to provide a novel saccharide primer which satisfies such conditions and a method for using the primer.

### Means for Solving the Problem

As shown in Examples described below, the present inventors have prepared a saccharide primer Lac-O-C13:1(2) represented by the following chemical formula (II)

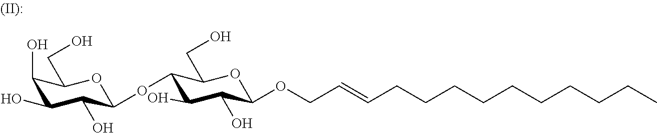

and administered the saccharide primer to mouse melanoma cells (B16 cells), resulting that, in the medium for culturing the B16 cells, extension of sugar chains equal to or greater than those obtained by using a conventional primer (i.e. saccharide primer Lac-O-C12 having no double bond in its alkyl chain) was observed. Further, the present inventors have found following facts: when the synthesized sugar chain existing in the medium is reacted with ozone, the double bond of the alkenyl chain of the above-described chemical formula (II) included in the synthesized sugar chain is cleaved by an oxidation reaction caused by the ozone, and as a result, an aldehyde group introduced into the cleaved position can make the sugar chain bind to a substrate. The present invention has been thus completed.

In other words, the saccharide primer according to the present invention is a saccharide primer having an alkenyl chain that has one or more double bonds in a position other than an alkyl chain terminal, and represented by the following general formula (I):

Lac-O-Ck:m(n) Formula (I)

(in the general formula (I), Ck represents an alkenyl chain having k of C atoms, m represents a total number of double bonds, and n represents a position of the double bond in the alkenyl chain (that is, in the case where C atoms are numbered from the C atom that binds directly to Lac-O, which is numbered as 1, a double bond between the nth C atom and the (n+1)th C atom is represented by n). Here, k is an integer in the range of 8 to 14, when m is 1, n is an integer satisfying 1≦n≦k−2, and when m satisfies 2≦m≦k−1, n is a plurality of integers satisfying 1≦n≦k−1).

Here, k is preferably 12 or 13.

In addition, a method for synthesizing a sugar chain, according to the present invention, is a method for synthesizing a sugar chain including: administering the saccharide primer to a sugar chain-synthesizing cell, and reacting the saccharide primer with the cell.

Further, a method, for oxidizing a sugar chain, according to the present invention is a method for oxidizing a sugar chain including steps of: synthesizing a sugar chain by the above-described method, and cooling the sugar chain synthesized in the foregoing step followed by adding ozone to the cooled sugar chain.

In addition, a method for producing a sugar chain array according to the present invention is a method for producing a sugar chain array including steps of: oxidizing a sugar chain by the above-described method, reacting the sugar chain oxidized in the foregoing step with a reducing agent, and binding the sugar chain reduced in the foregoing step to a substrate. The sugar chain array produced by this method is also within the scope of the present invention.

Further, a method for synthesizing a glycopeptide according to the present invention is a method for synthesizing a glycopeptide including steps of: oxidizing a sugar chain by the above-described method, reacting the sugar chain oxidized in the foregoing step with a reducing agent, and reacting the sugar chain reduced in the foregoing step with a peptide. The glycopeptides synthesized by this method is also within the scope of the present invention.

A “sugar chain-synthesizing cell” described herein means a cell having a biosynthesis pathway of a sugar chain, and a type of such a cell is not particularly limited.

A novel saccharide primer which makes it possible to efficiently synthesize a sugar chain and to easily use the synthesized sugar chain for various purposes, and a method for using the primer can be provided by the present invention.

### CROSS REFERENCE TO RELATED DOCUMENTS

The present application claims the benefit of priority based on Japanese Patent Application No. 2007-325804 filed on Dec. 18, 2007, which is herein incorporated by reference.

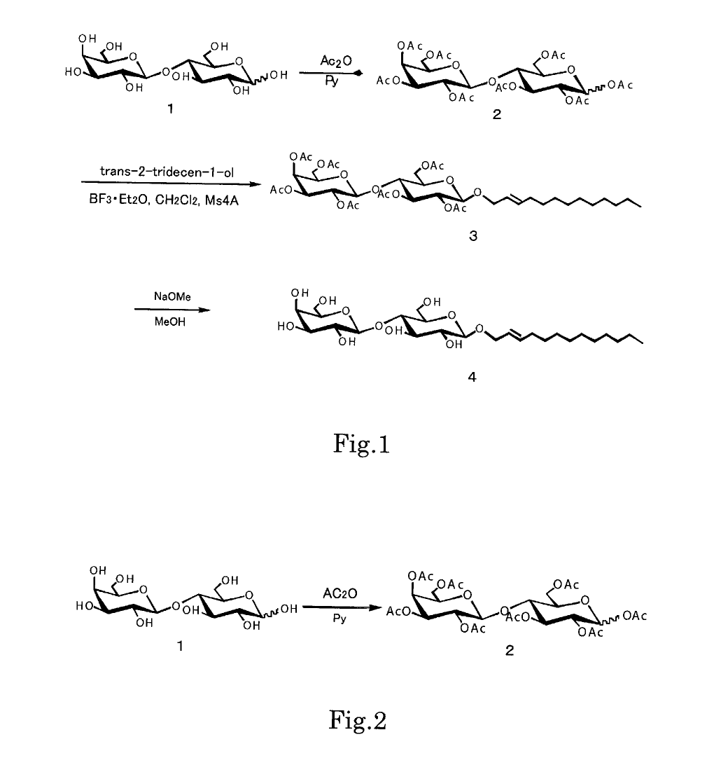

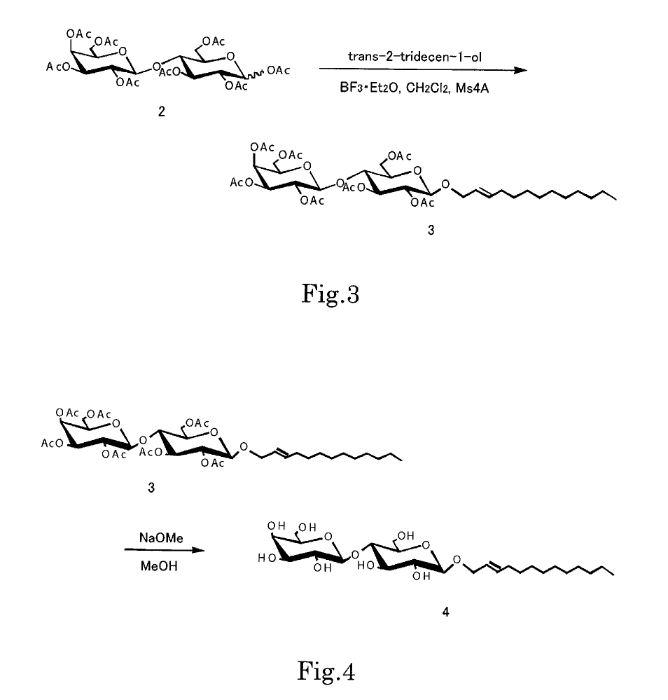

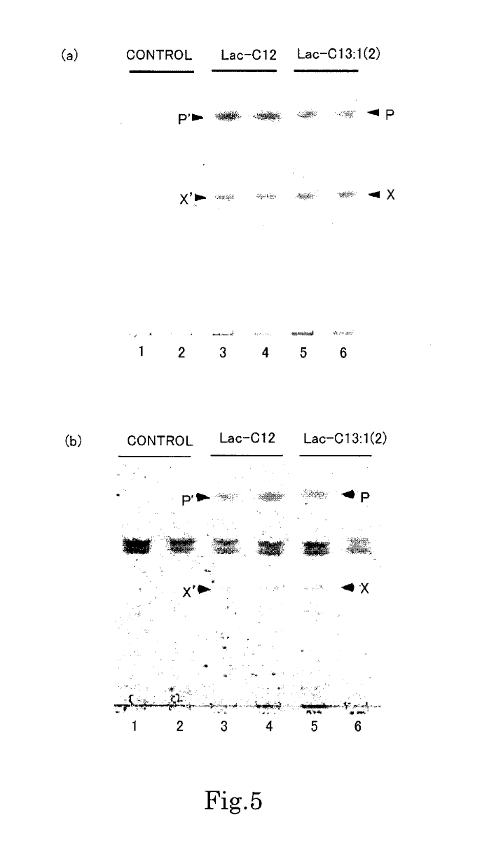

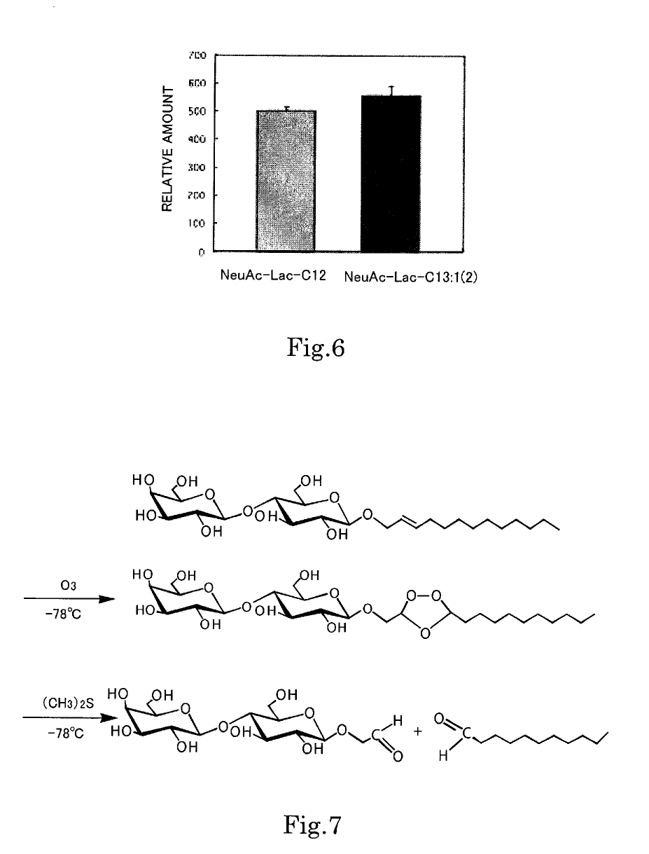

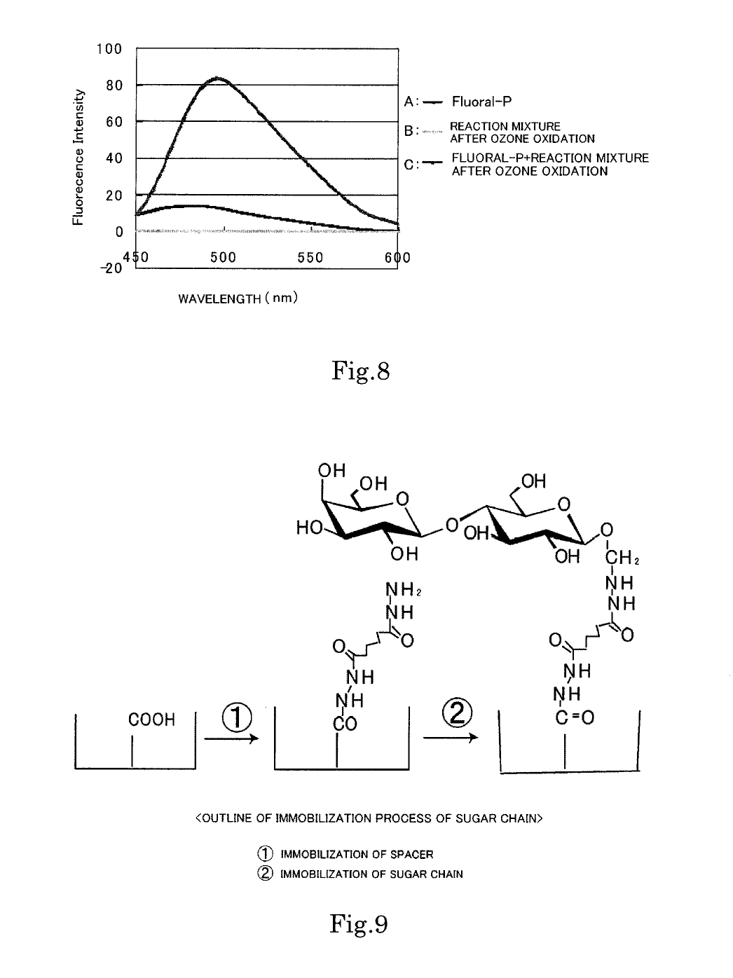

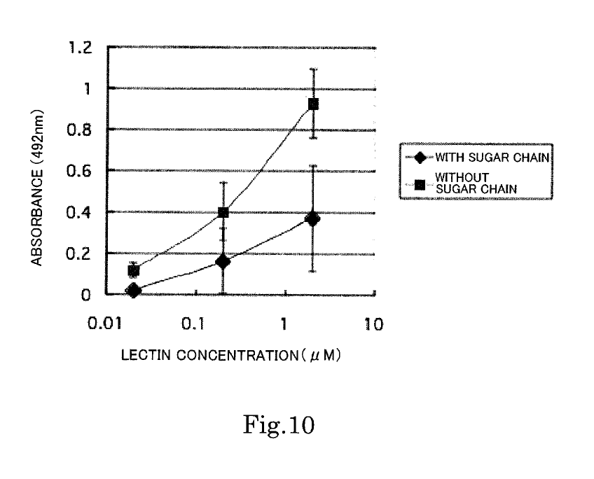

### BRIEF DESCRIPTION OF DRAWINGS

[FIG. 1] FIG. 1 is a synthesis scheme of 1-O-(trans-2-tridecenyl)-4-O-(β-D-galactopyranosyl)-β-D-glucopyranoside in an Example of the present invention.

[FIG. 2] FIG. 2 is a synthesis scheme of lactose octaacetate (Ac-Lac) in an Example of the present invention.

[FIG. 3] FIG. 3 is a synthesis scheme of 1-O-(trans-2-tridecenyl)-4-O-(2,3,4,6-tetra-O-acetyl-β-D-galactopyranosyl)-2,3,6-tri-O-acetyl-D-glucopyranoside (Ac-Lac-C13:1(2)) in an Example of the present invention.

[FIG. 4] FIG. 4 is a synthesis scheme of 1-O-(trans-2-tridecenyl)-4-O-(β-D-galactopyranosyl)-D-glucopyranoside (Lac-C13:1(2)) in an Example of the present invention.

[FIG. 5] FIG. 5 is a view showing results of analysis of products produced upon administration of saccharide primers to B16 cells, obtained by using HPTLC, in an Example of the present invention. (a) shows a medium fraction and (b) shows a cell fraction. Lanes 1 and 2 show controls, lanes 3 and 4 show saccharide primers Lac-C12, and lanes 5 and 6 show saccharide primers Lac-C13:1(2). Both (a) and (b) show results of resorcinol staining.

[FIG. 6] FIG. 6 is a graph showing results of quantitation of glycolipids, obtained by using a densitometer, in an Example of the present invention. Amounts of products having an extended sugar chain in the medium fraction were compared.

[FIG. 7] FIG. 7 is an ozone oxidation reaction scheme of a saccharide primer Lac-C13:1(2) in an Example of the present invention.

[FIG. 8] FIG. 8 is a graph showing fluorescence intensity of each sample in an Example of the present invention. The excitation wavelength is 410 nm.

[FIG. 9] FIG. 9 is a diagram showing an outline of an immobilization process of a sugar chain in an Example of the present invention.

[FIG. 10] FIG. 10 is a graph showing binding of ECA lectin to lactose when a sugar chain was immobilized in PBS in an Example of the present invention.

### BEST MODES FOR CARRYING OUT THE INVENTION

Unless otherwise described in modes and Examples, methods described in standard protocols, or modified and altered methods thereof are used. In addition, when commercially available reagent kits or measurement devices are used, protocols attached thereto are used unless otherwise described.

The object, feature, advantage and idea of the present invention are obvious to a person skilled in the art based on the present description, and the person skilled in the art can easily reproduce the present invention based on the present description. Modes, specific Examples and the like described below show preferred embodiments of the present invention and are shown for the purpose of illustration or explanation, which do not limit the present invention thereto. It is obvious to the person skilled in the art that various modifications can be performed based on the present description within the intention and the scope of the present invention disclosed in the present description.

### Novel Saccharide Primer

The saccharide primer according to the present invention is a saccharide primer having an alkenyl chain that includes one or more double bonds in a position other than the alkyl chain terminal, and represented by the following general formula (I):

Lac-O-Ck:m(n) Formula (I)

In the general formula (I), Ck represents an alkenyl chain having k of C atoms, m represents a total number of double bonds, and n represents a position of the double bond in the alkenyl chain (that is, in the case where C atoms are numbered from the C atom that binds directly to Lac-O, which is numbered as 1, a double bond between the nth C atom and the (n+1)th C atom is represented by n). Here, Ck may be a linear alkenyl chain or a branched alkenyl group, however, it is preferable that it be a linear alkenyl chain. In addition, although k is not particularly limited, k is preferably an integer in the range of 8 to 14, particularly 12 or 13. Further, when m is 1, n is an integer satisfying 1≦n≦k−2. Furthermore, when m satisfies 2≦m≦k−1, n is a plurality of integers satisfying 1≦n≦k−1, and it is particularly preferable that the integers include 2 (for example, n=2 or n=2 and 5, or the like).

In the general formula (I), —O-Ck preferably binds to a carbon atom at the 1-position of D-glucose that constitutes Lac (lactose).

In the general formula (I), the bond between Lac and —O-Ck may be an α-bond or a β-bond. Therefore, when used as a saccharide primer, the primer may contain either one or both of an α-anomer having an α-bond and a β-anomer having a β-bond. In the case where the primer is a mixture of the above, the mixture ratio is not particularly limited.

As an example of such a saccharide primer, a saccharide primer Lac-O-C13:1(2), represented by the following chemical formula (II), was used in Examples.

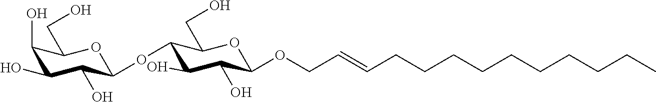

### Method for Synthesizing Saccharide Primer

The above-described saccharide primer can be synthesized with a known technique to a person skilled in the art. For example, lactose, which can be a sugar chain part of the saccharide primer (corresponding to 1 in FIG. 1), is reacted with acetic anhydride in the presence of a pyridine solvent to synthesize lactose octaacetate (corresponding to 2 in FIG. 1). Then, glycosylation reaction of alcohol having a double bond, such as trans-2-tridecen-1-ol, with lactose octaacetate in a dichloromethane solvent under anhydrous conditions (corresponding to 3 in FIG. 1), and subsequent deacetylation give the desired saccharide primer (corresponding to 4 in FIG. 1). There is no particular limitation for the carbon number of the alcohol used here, however, a monohydric alcohol having a carbon number in the range of 8 to 14 is preferable, and a monohydric alcohol having a carbon number of 12 or 13 is more preferable.

In addition, the saccharide primer may be synthesized by a method such as an enzymatic method.

### Method for Synthesizing Sugar Chain

It is known that, in general, when a saccharide primer is administered to a sugar chain-synthesizing cell which has a biosynthesis pathway of a sugar chain, this saccharide primer enters into the cell and reacts with a glycosyltransferase in the cell, and as a result, sugar chain extension occurs.

As shown in the Examples described below, when the saccharide primer of the present invention is reacted with a sugar chain-synthesizing cell, sugar chains can be efficiently synthesized.

Here, examples of the sugar chain-synthesizing cell include eukaryotic cells having a gene involved in sugar chain synthesis, such as mammalian cells, insect cells, plant cells, and yeasts.

Cultured cells may be primary cultured cells or established cultured cells. The cultured cells include all kinds of cells as long as they are cells cultured under culture conditions, and are not limited by the number of passages in culture, culture time, the number of transfers, and the like. Examples of the established cultured cells include simian renal cells COST, human leukemia-derived cells HL60, canine renal cells MDCK and the like. It is particularly preferable to use mouse melanoma cells (for example, B16 cells or the like).

Cells taken from living organisms are preferably proliferating cells, and particularly cancer cells.

When a saccharide primer is administered to the sugar chain-synthesizing cell described above, this cell reacts with the saccharide primer, and as a result, a desired sugar chain can be obtained. Specifically, a saccharide primer is administered at a concentration of 50 μM to a medium in which sugar chain-synthesizing cells (for example, B16 cells) are cultured, and the mixture is incubated at 37° C. for about 2 days. During that period, the saccharide primer is introduced into the cells, a sugar is added to the saccharide primer, and various types of sugar chains are produced in the culture liquid. The sugar chains are recovered from the culture liquid by a method known to a person skilled in the art, for example, a lectin column or a commercially available sugar chain purification kit.

In the case where the number of the types of synthesized sugar chains is desired to be increased, it is sufficient to increase combinations of saccharide primers and sugar chain-synthesizing cells by increasing the number of the types of sugar chain-synthesizing cells and the saccharide primers to be used.

### Introduction of Aldehyde Group

The saccharide primer of the present invention has an alkenyl chain that contains one or more double bonds in a position other than the alkyl chain terminal. Therefore, when this saccharide primer is administered to a sugar chain-synthesizing cell to synthesize a sugar chain, a sugar chain having an alkenyl chain that contains a double bond in the alkyl chain can be synthesized. When this double bond is reacted with ozone, which is a potent oxidizing agent conducting an oxidation reaction that breaks a carbon-carbon double bond in an organic compound, the synthesized sugar chain can be efficiently oxidized. As ozone can be generated by introducing oxygen into an ozone generator, which utilizes silent discharge or the like, it becomes possible to oxidize sugar chains easily and at a low cost.

The way of the oxidation reaction caused by ozone is not particularly limited as long as the function of the synthesized sugar chain is maintained, and can be performed with a technique known to a person skilled in the art (for example, see F. Helling, A. Shang, M. Calves, S. Zhang, S. Ren, R. K. Yu, H. F. Oettgen, and P. O. Livingston, Cancer Res 54, 197-203 (1994)). Specifically, an oxidation reaction can be performed as follows: dissolving a synthesized sugar chain in a solvent such as methanol, dichloromethane, acetic acid, and the like; cooling the resultant solution to −78° C.; and blowing (bubbling) a 2% ozone gas generated with an ozone generator into the solution. Ozone does not produce organochlorine compounds, in this reaction, and thus, by using ozone in the oxidation reaction of sugar chains synthesized with the saccharide primers, unlike conventional oxidation reactions, it becomes possible to obtain highly pure sugar chain oxides with a low impurity content.

When the sugar chain thus oxidized is reacted with a reducing agent (for example, dimethylsulfide, zinc (when in an acetic acid solvent), triphenylphosphine and the like), an aldehyde group (—CH═O) can be introduced into a position where the double bond was present in the alkyl group in the synthesized sugar chain. Since the introduced aldehyde group has a strong reducing property and is highly reactive, the synthesized sugar chain can be widely used through a variety of subsequent reactions. In addition, since the terminal alkyl chain is simultaneously removed, it is possible to remove, from a sugar chain, a hydrophobic moiety which may hamper subsequent use of the sugar chain.

When a plurality of double bonds are present in the alkyl group in the synthesized sugar chain, the removal of the alkyl chain occurs from an alkenyl group, which is on the closest position to the sugar moiety in the sugar chain, by completely reacting the sugar chain with the reducing agent.

### Method for Producing Sugar Chain Array

The sugar chain into which the aldehyde group is introduced can be easily and firmly bound to an electrically charged substrate or the like, without using an organic solvent or the like. Therefore, a sugar chain array can be efficiently produced by using a substrate comprising, for example, glass, polystyrene, gold and the like.

In addition, the sugar chain into which the aldehyde group is introduced can be used for a sugar chain array obtained by immobilization on a solid-phase support or the like.

As described above, the sugar chain into which the aldehyde group is introduced does not have the long hydrophobic group. Therefore, non-specific adsorption or binding of objects to be detected can be suppressed by using the sugar chain array. Accordingly, the sugar chain array produced in accordance with the method of the present invention allows more accurate analysis of an interaction between a sugar chain and a substance that interacts with the sugar chain.

### Method for Synthesizing Glycopeptide

Since an aldehyde group binds to an amino group to provide an amide group, a glycopeptide can be synthesized by reacting a sugar chain into which an aldehyde group is introduced with an amino group that is a side chain of a peptide. For a binding reaction of a sugar chain to a peptide, it is sufficient to use a technique known to a person skilled in the art (for example, see F. Helling, A. Shang, M. Calves, S. Zhang, S. Ren, R. K. Yu, H. F. Oettgen, and P. O. Livingston, Cancer Res 54, 197-203 (1994)).

### EXAMPLES

Hereinafter, the embodiments described above are specifically described by using Examples, however, such Examples are for illustrative purposes and do not limit the present invention thereto.

### Example 1

### Synthesis of Novel Saccharide Primer

The saccharide primer, 1-O-(trans-2-tridecenyl)-4-O-(β-D-galactopyranosyl)-P-D-glucopyranoside, of the following chemical formula (II) was designed and synthesized as follows.

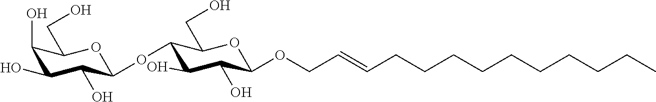

The total synthesis is shown in FIG. 1 as a scheme.

First, lactose (corresponding to 1 in FIG. 1) was reacted with acetic anhydride in the presence of a pyridine solvent to synthesize lactose octaacetate (corresponding to 2 in FIG. 1). Subsequently, glycosylation reaction of trans-2-tridecen-1-ol with lactose octaacetate in a dichloromethane solvent under anhydrous conditions gave 1-O-(trans-2-tridecenyl)-4-O-(2,3,4,6-tetra-O-acetyl-β-D-galactopyranosyl)-2,3,6-tri-O-acetyl-β-D-glucopyranoside (corresponding to 3 in FIG. 1). Finally, 1-O-(trans-2-tridecenyl)-4-O-(2,3,4,6-tetra-O-acetyl-(3-D-galactopyranosyl)-2,3,6-tri-O-acetyl-β-D-glucopyranoside was deacetylated to give the desired compound, 1-O-(trans-2-tridecenyl)-4-O-(β-D-galactopyranosyl)-β-D-glucopyranoside (corresponding to 4 in FIG. 1).

Details of each synthesis step are described below. Hereinafter, lactose octaacetate is described as “Ac-Lac”, 1-O-(trans-2-tridecenyl)-4-O-(2,3,4,6-tetra-O-acetyl-β-D-galactopyranosyl)-2,3,6-tri-O-acetyl-β-D-glucopyranoside is described as “Ac-Lac-C13:1(2)”, and 1-O-(trans-2-tridecenyl)-4-O-(β-D-galactopyranosyl)-β-D-glucopyranoside is described as “Lac-C13:1(2)”.

### (1) Synthesis of Ac-Lac

An overall scheme for synthesizing Ac-Lac is shown in FIG. 2.

First, 1.7 g of lactose (SIGMA), which is described as 1 in FIG. 2, was dissolved in 35 ml of dehydrated pyridine (Wako), and 20 ml of acetic anhydride (Wako) was added dropwise to the resultant solution being stirred in an ice bath. After 35 hours, completion of the reaction was confirmed with thin layer chromatography (TLC). Then, 20 ml of ethanol was added dropwise, and the resultant solution was stirred for 30 minutes at room temperature to stop the reaction. The solvent was evaporated under reduced pressure. The resultant residue was dissolved in ethyl acetate, and the organic layer was washed with 1N HCl twice, saturated aqueous NaHCO₃ twice, H₂O once, and brine twice. Na₂SO₄ was added, and the resultant mixture was vigorously stirred for 3 hours to be dehydrated. Subsequently, Na₂SO₄ was filtered off by cotton filtration, and the solvent was removed under reduced pressure. The residue was purified with silica gel column chromatography (internal diameter is 3 cm, length is 20 cm, hexane/ethyl acetate=2/3) to give Ac-Lac (corresponding to 2 in FIG. 2). The compound was confirmed by ¹H-NMR and MALDI-TOF/MS analyses. The results are shown below.

### Results

Yield (weight): 2.84 g (4.19 mmol)
Yield (%): 88.20
Analysis

¹H-NMR (300 MHz, solvent: CDCl₃)

δ (ppm): 6.70-5.64 (d, 1H, Glc-1), 5.47-4.90 (m, 5H, Gal-H₄, Gal-H₃, Gal-H₂, Glc-H₃, Glc-H₂), 4.47-4.41 (m, 2H, Gal-H₁, Gal-H₆), 4.15-4.02 (m, 3H, Glc-H₆, Gal-H₅, Gal-H₅), 3.87-3.69 (m, 3H, Glc-H₄, Glc-H₅, Glc-H₅), 2.16-1.97 (m, 24H, acetyl group)

It was found from the J values that the peak at 6.25 ppm is assigned to the α-anomer and the peak at 5.64 ppm is assigned to the β-anomer. From the ratio of these peak areas, the ratio of the products, α-anomer to β-anomer, was found to be about 1:4.
MALDI-TOF-MS Exact Mass: 678.2
[M+Na]⁺ calculated 701.19, measured 701.35
[M+K]⁺ calculated 717.16, measured 717.43

### (2) Synthesis of Ac-Lac-C13:1(2)

The scheme for synthesizing Ac-Lac-C13:1(2) is shown in FIG. 3.

Since this reaction is performed under anhydrous conditions, a 10 ml syringe and a syringe needle were placed in a dryer at 60° C. on the day before the experiment. Further, on the day before the experiment, molecular sieves 4A (Wako, powder, MS4A) and a stirrer were put into a four-necked flask, and heated together with to 200° C. in an oven. After heating, the container was dried in a desiccator until cooled down to room temperature, and thus the equipments were dried and MS4A was activated.

To the four-necked flask containing the activated MS4A therein, 2.15 g of Ac-Lac (corresponding to 2 in FIG. 3) was added, and nitrogen substitution in the reaction system was performed. To the flask, 42.5 ml of dehydrated dichloromethane (Wako) was added to give a solution, and then 1.25 g of trans-2-tridecen-1-ol (AVOCADO, 17753) and 0.2 ml of BF₃.Et₂O (Wako, 022-08362) were added. The mixture was stirred at room temperature. After 2.5 hours, additional 0.3 ml of BF₃.Et₂O was added. After 5 hours, 1 ml of triethylamine (Wako) was added for neutralization and the reaction was quenched. The reaction mixture was filtered with Celite (trademark), dried under vacuum overnight, and subsequently purified with silica gel column chromatography (internal diameter 6 cm×length 20 cm, hexane/ethyl acetate=1/1) to give the desired compound (corresponding to 3 in FIG. 3, Ac-Lac-C13:1(2)). At this stage, the product was composed of β-anomer only. The obtained compound was confirmed by ¹H-NMR and MALDI-TOF/MS analyses. The results are shown below.

### Results

Yield (weight): 500 mg (0.61 mmol)
Yield (%): 20.3%
Analysis

¹H-NMR (300 MHz, solvent: CDCl₃)

δ(ppm): 5.70-5.61 (m, 1H, —CH═CH—C₁₀H₂₁), 5.47-5.38 (m, 1H, —CH═CH—C₁₀H₂₁), 5.33-5.32 (t, 1H, Gal-H₄), 5.20-5.05 (t, dd, 2H, Glc-H₃, Gal-H₂), 4.95-4.85 (dd, dd, 2H, Glc-H₂, Gal-H₃), 4.50-4.44 (m, 3H, Glc-H₁, Gal-H₁, Gal-H₆), 4.14-4.00 (m, 5H-CH₂—CH═CH—, Glc-H₆, Gal-H₅, Gal-H₆), 3.86-3.74 (m, 2H, Glc-H₄, Glc-H₆), 3.58-3.57 (m, 1H, Glc-H₅), 2.13-1.98 (m, 23H, —CH═CH—CH₂—, acetyl group), 1.30-1.21 (m, 16H, —CH═CH—CH₂—C₈H₁₆—CH₃), 0.88-0.83 (t, 3H, —CH═CH—C₉H₁₈—CH₃)

MALDI-TOF-MS Exact Mass: 816.4

[M+Na]⁺ calculated 839.37, measured 839.11

[M+K]⁺ calculated 855.34, measured 855.20

### (3) Synthesis of Lac-C13:1(2)

The scheme for synthesizing Lac-C13:1(2) is shown in FIG. 4.

First, 0.5 g of Ac-Lac-13:1(2) (corresponding to 3 in FIG. 4) was dissolved in 15 ml of NeOH (Nacalai Tesque), a few drops of 28% MeONa/MeOH solution (Wako, 197-02463) were added with a Pasteur pipette, and the mixture was stirred. In order to progress the reaction, a few drops of MeONa/MeOH were added 2.5 hours later, and 3.5 hours later as well. After 5.5 hours, the completion of the reaction was confirmed with TLC, and an activated Amberlite (cation exchange resin, ORGANO) was added for neutralization of the reaction mixture. The Amberlite was removed by cotton filtration, and the solvent was removed off under reduced pressure to give Lac-C13:1(2) (corresponding to 4 in FIG. 4). The compound was confirmed by ¹H-NMR and MALDI-TOF/MS analyses. The results are shown below.

### Results

Yield (weight): 300 mg (0.57 mmol)
Yield (%): 93.8% (the overall yield was 19.0%)
Analysis

¹H-NMR (300 MHz, solvent: CD₃OD)

δ(ppm): 5.70-5.61 (m, 1H, —CH═CH—C₁₀H₂₁), 5.54-5.45 (m, 1H, —CH═CH—C₁₀H₂₁), 1.97-1.95 (q, 2H, —CH═CH—CH₂—C₉H₁₉), 1.29-1.19 (m, 16H, —CH═CH—CH₂—C₈H₁₆—CH₃), 0.82-0.78 (t, 3H, —CH═CH—C₉H₁₉—CH₃)

MALDI-TOF-MS Exact Mass: 522.3

[M+Na]⁺ calculated 545.29, measured 545.02

[M+K]⁺ calculated 561.27, measured 560.98

### Example 2

### Sugar Chain Extension Reaction in B16 Cells with Novel Saccharide Primer

In the present Example, a sugar chain extension reaction in B16 cells was performed with the saccharide primer Lac-C12 (a), or the saccharide primer Lac-C13:1(2) (b), which was synthesized in Example 1, and the results were measured.

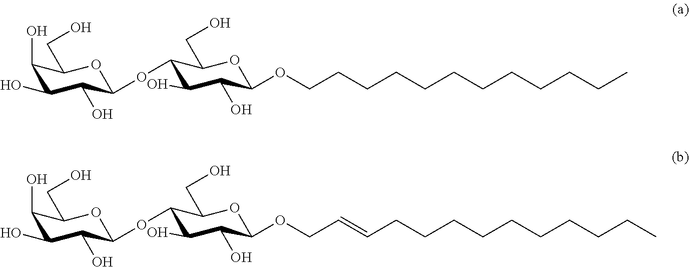

### (1) Sugar Chain Extension Reaction in Living Cells

First, the saccharide primer Lac-C12 and the saccharide primer Lac-C13:1(2) were added to each medium in which B16 cells (mouse melanoma cells strongly expressing GM3) (Japan Health Science Foundation, Health Science Research Resources Bank) were plated at a concentration of 2.0×10⁶ cells/plate, and then incubated for 48 hours. Then, the medium and the cells were separated by centrifugation, and each of collected substances was analyzed with HPTLC. The results are shown in FIG. 5.

In the cells to which the saccharide primer Lac-C13:1(2) was administered, a product X was confirmed in both the medium fraction and the cell fraction. Similarly, in the cells to which the saccharide primer Lac-C12 was administered, a product X′ was confirmed in both the medium fraction and the cell fraction.

(2) Mass Spectrometry Analysis with MALDI-TOF-MS

The product obtained by the administration of the saccharide primer Lac-C13:1(2) was isolated and purified by TLC blotting, and subjected to mass spectrometry analysis with MALDI-TOF-MS. It was found that the peak of X is m/z=812.44 and that X is the saccharide primer to which one molecule of sialic acid was added.

In addition, the extended sugar chain product X was also analyzed based on MALDI-PSD spectra.

In the obtained PSD spectra, the peak of sodium adduct of the primer (Lac-C13:1(2)+Na=545.3) and NeuAc+anGal+Na=476.1 were confirmed, and NeuAc+Glc-C13:1(2)+Na=649.3 was not present. Therefore, the product was determined to be NeuAc-Gal-Glc-C13:1(2), that is, NeuAc-Lac-C13:1(2).

(3) Comparison of Sugar Chain Extension Efficiency of the Saccharide Primer Lac-C13:1(2) with that of the Saccharide Primer Lac-C12

With regard to the saccharide primer Lac-C12 and the saccharide primer Lac-C13:1(2), their sugar chain extension efficiencies in B16 cells and amounts of the saccharide primers taken into the cells were compared by a quantitative analysis with a densitometer.

The results were shown in FIG. 6. The amounts of the extended sugar chain products in respective medium fractions were compared, and it was found that the sugar chain extension efficiency of the saccharide primer Lac-C13:1(2) was similar to or greater than that of the saccharide primer Lac-C12.

### Example 3

### Ozone Oxidation Reaction of Novel Saccharide Primer

### (1) Ozone Oxidation

The scheme for an oxidation reaction of the saccharide primer Lac-C13:1(2) with ozone is shown in FIG. 7.

With regard to the experimental procedure of the ozone oxidation described below, the present inventors have referred to the reference (F. Helling, A. Shang, M. Calves, S. Zhang, S. Ren, R. K. Yu, H. F. Oettgen, and P. O. Livingston, Cancer Res 54, 197-203 (1994)).

To 1 mg of the saccharide primer Lac-C13:1(2) (Mw=522.63), 2 ml of methanol (Nacalai Tesque) was added and the primer was completely dissolved by ultrasonic irradiation. Then, a test tube containing the sample therein was put into an ethanol/dry ice bath, and cooled to −78° C. Ozone was generated by using an ozone generator (Ecodesign Inc., air-cooled type, ED-OG-A10) and an oxygen tank (Toyoko Kagaku Co., Ltd., the capacity is 1.5 m³). The pressure of the oxygen tank was set to 0.1 MPa, the flow rate was set to 50 ml/min, and bubbling was performed in the sample for 25 minutes. After the completion of the reaction was confirmed with TLC, bubbling of nitrogen gas was performed for about 30 minutes to remove excess ozone from the reaction mixture. Since it was assumed that a peroxide was present in the reaction mixture, 100 μl of dimethylsulfide (Wako) was added as a reducing agent to reduce the peroxide to aldehyde. The resultant mixture was stirred vigorously for 30 minutes at −78° C., and then for 90 minutes at room temperature. Then 10 μl of the reaction mixture was put aside for HPTLC analysis, and the rest of the reaction mixture was transferred to a 15 ml centrifuging tube and stored at 4° C.

(2) Mass Spectrometry Analysis with MALDI-TOF-MS

The obtained product was isolated and purified by TLC blotting, and subjected to mass spectrometry analysis with MALDI-TOF-MS. Since m/z=407.16 was detected, the product was found to be Lac-CH₂—CHO.

### (3) Detection of an Aldehyde Group by Fluoral-P

As shown in FIG. 8, a solution C, which had been given by mixing Fluoral-P and the reaction mixture after the ozone oxidation, exhibited obviously stronger fluorescence intensity than those exhibited by a solution A composed of Fluoral-P only and a solution B composed of the reaction mixture after the ozone oxidation only. From this result, it was shown that the aldehyde was generated in the reaction mixture by the ozone oxidation and that the fluorescence was excited by the reaction of the aldehyde with Fluoral-P.

### Example 4

### Immobilization of Ozone-Oxidized Saccharide Primer to Substrate and Sugar Chain Recognition

An outline of an immobilization process of the sugar chain is shown in FIG. 9.

### (1) Immobilization of the Sugar Chain to a Substrate

### (i) Immobilization of a Spacer

An ELISA plate providing a carboxyl group (96 wells, MS-8796F, Sumitomo Bakelite Co., Ltd.) was used as a substrate for immobilization. And adipic dihydrazide (Nacalai Tesque, No. 01048-12) was used as a spacer providing an amino group.

Further, as an activating reagent used in the immobilization of the spacer, EDC (1-ethyl-3-(3-dimethylaminopropyl)carbodiimide HCl; PIERCE Biotechnology 22980), NHS (N-hydroxysuccinimide; Nacalai Tesque, 189-14) were used. In addition, as a running buffer for the entire steps of the reaction, a solution, which had been prepared by dissolving 9.6 g of PBS (−) (Nissui Pharmaceutical Co., Ltd.) in MilliQ such that the amount was adjusted to be 1 L, was used. The experimental method was as follows.

First, the following samples were prepared.
a) Pretreatment solution for the immobilization reaction: prepared at a ratio of 200 mM acetic acid buffer/EtOH=2/1
b) Activating reagent solution: prepared at a ratio of 730 mM EDC aqueous solution/183 mM NHS solution=1/1
c) Spacer solution: 129 mM adipic dihydrazide aqueous solution

Next, the spacer was immobilized.

To the plate, 60 μL of the pretreatment solution for the immobilization reaction was injected, and then 10 μL of the activating reagent solution was injected. The resultant solution was shaken for 15 minutes at room temperature (in order to activate the functional group on the plate). Next, 35 μL of the spacer solution was injected (final concentration: 29 mM), and then the plate was gently shaken by hand and left for one hour at room temperature. Finally, the solution was removed and then washed with MilliQ 5 times. An amino group was thus provided by the substrate.

### (ii) Immobilization of the Ozone-Oxidized Saccharide Primer

As a sugar chain used for immobilization, Lac-CH₂—CHO, which had been produced by oxidizing the sugar obtained by the primer Lac-C13:1(2), was used. In addition, as Lac-CH₂—CHO, the reaction mixture after the ozone oxidation reaction was used without purifying. As an oxidizing agent, sodium cyanoborohydride NaCNBH₃ (Aldrich) was used, and 1N hydrochloric acid was used to make the solution acidic. The experimental method was as follows.

First, the following samples were prepared.
d) Pretreatment solution for the immobilization reaction: PBS (−) or MeOH
e) Lac-CH₂—CHO/MeOH solution or NeuAc-Lac-CH₂—CHO/MeOH solution
f) 5% NaCNBH₃/PBS (−) or MeOH solution
g) 1N hydrochloric acid

Next, 40 μL of the pretreatment solution for the immobilization reaction, 10 μL of Lac-CH₂—CHO/MeOH solution, 0.5 μL of 1N hydrochloric acid, and 10 μL of 5% NaCNBH₃ were injected. The resultant solution was shaken overnight at 4° C., and then washed with PBS (−) 5 times. The sugar chain was thus bound to the amino group of the spacer provided by the substrate, and the sugar chain was immobilized to the substrate.

### (2) Evaluation of Lactose Recognition by an Enzyme Labeling Method

In order to confirm that Lac-CH₂—CHO was immobilized on the substrate and that the sugar chain can specifically bind to a protein, a biotin-labeled ECA lectin (Seikagaku Corporation, No. 300418), which is a lectin that recognizes lactose and binds thereto, was used. Streptavidin Peroxidase Conjugate (CALBIOCHEM, No. 189733) was used as an enzyme-labeled avidin and then detected by ELISA method. The immobilization reaction was performed in a PBS solvent and the relationship between the lectin concentrations and the absorbances was shown in FIG. 10.

As a result, it was shown that the absorbances increased depending on the lectin concentrations and thus the sugar chain Lac-CH₂—CHO was immobilized on the substrate.

1. A saccharide primer comprising an alkenyl chain having one or more double bonds, and represented by the following general formula (I):
Lac-O-Ck:m(n), Formula (I)
wherein Ck represents an alkenyl chain having k of C atoms, m represents a total number of double bonds, and n represents a position of the double bond in the alkenyl chain; k is an integer in the range of 8 to 14; when m is 1, n is an integer satisfying 1≦n≦k−2; and when m satisfies 2≦m≦k−1, n is a plurality of integers satisfying 1≦n≦k−1.

2. A method for synthesizing a sugar chain comprising:
administering the saccharide primer according to claim 1 to a sugar chain-synthesizing cell; and
reacting the cell with the saccharide primer.

3. A method for oxidizing a sugar chain comprising:
synthesizing the sugar chain by the method according to claim 2; and
reacting the synthesized sugar chain with ozone.

4. A method for producing a sugar chain array comprising:
oxidizing the sugar chain by the method according to claim 3;
reacting the oxidized sugar chain with a reducing agent; and
binding the reduced sugar chain to a substrate.

5. A method for synthesizing a glycopeptide comprising:
oxidizing a sugar chain by the method according to claim 3;
reacting the oxidized sugar chain with a reducing agent; and
reacting the reduced sugar chain with a peptide.

6. A sugar chain array produced by the method according to claim 4.

7. A glycopeptide synthesized by the method according to claim 5.
